(19) United States
(12) Patent Application Publication
(10) Pub. No.: US 2022/0118138 A1
(43) Pub. Date: Apr. 21, 2022

(54) Portable Sanitizing Station for Spa or Salon Tools

(71) Applicant: Angela Lockhart, Dallas, TX (US)

(72) Inventor: Angela Lockhart, Dallas, TX (US)

(21) Appl. No.: 17/503,472

(22) Filed: Oct. 18, 2021

Related U.S. Application Data
(60) Provisional application No. 63/093,479, filed on Oct. 19, 2020.

(51) Int. Cl. A61L 2/10

(52) U.S. Cl. CPC A61L 2/10; A61L 2202/11; A61L 2202/24; A61L 2202/122; A61L 2202/16; A61L 2202/121

(57) ABSTRACT

A portable sanitizing station for receiving one or more spa or salon tools may include a body portion having an opening wherein the one or more spa or salon tools are inserted into the body portion, a lid having a closure mechanism that secures the one or more spa or salon tools within the body portion, an ultraviolet (UV) light mechanism within the body portion that sanitizes the one or more spa or salon tools to kill bacteria, germs, viruses, and/or pathogens that may be present on the one or more spa or salon tools before they are used in a spa or salon, a button on an exterior wall of the sanitizing station, wherein the button is pressed to activate the UV light mechanism, and a handle and at least one strap to transport the sanitizing station.

### CROSS REFERENCE TO RELATED APPLICATION

This application claims the benefit under 35 U.S.C. § 119(e) of U.S. Provisional Patent Application Ser. No. 63/093,479 filed on Oct. 19, 2020, entitled “Illums,” which is incorporated by reference in its entirety.

### FIELD OF THE DISCLOSURE

The present disclosure generally relates to sanitization, and more particularly to a portable sanitizing station for spa and salon tools.

### BACKGROUND

Spas and salons sometimes may not properly disinfect their equipment, ultimately leading to the spread of harmful airborne pathogens like germs, viruses, and bacteria. Nail technicians may use buffers on the hands and feet of different clients without properly sanitizing the tools. Patrons also may be unable to verify if the salon is using proper sanitizing procedures before visiting.

### SUMMARY

Embodiments of the present disclosure may provide a portable sanitizing station for receiving one or more spa or salon tools, the sanitizing station comprising a body portion having an opening wherein the one or more spa or salon tools may be inserted into the body portion; a lid having a closure mechanism that may secure the one or more spa or salon tools within the body portion; and an ultraviolet (UV) light mechanism within the body portion that may sanitize the one or more spa or salon tools to kill bacteria, germs, viruses, and/or pathogens that may be present on the one or more spa or salon tools before they are used in a spa or salon. The sanitizing station may be in a bag form. The closure mechanism may be a zipper. A handle and/or one or more straps may be secured to the sanitizing station to transport the sanitizing station. A button may be provided on an exterior wall of the sanitizing station, wherein the button may be pressed to activate the UV light mechanism. A power supply may provide power to the UV light mechanism. The UV light mechanism may be rechargeable. The UV light mechanism may be recharged through a standard electrical outlet connection or a battery.

Other embodiments of the present disclosure may provide a portable sanitizing station for receiving one or more spa or salon tools, the sanitizing station comprising a body portion having an opening wherein the one or more spa or salon tools may be inserted into the body portion; a lid having a closure mechanism that may secure the one or more spa or salon tools within the body portion; an ultraviolet (UV) light mechanism within the body portion that may sanitize the one or more spa or salon tools to kill bacteria, germs, viruses, and/or pathogens that may be present on the one or more spa or salon tools before they are used in a spa or salon; and a button on an exterior wall of the sanitizing station, wherein the button may be pressed to activate the UV light mechanism. A power supply may provide power to the UV light mechanism, and the UV light mechanism may be rechargeable. The UV light mechanism may be recharged through a standard electrical outlet connection or a battery.

Further embodiments of the present disclosure may provide a portable sanitizing station for receiving one or more spa or salon tools, the sanitizing station comprising: a body portion having an opening wherein the one or more spa or salon tools may be inserted into the body portion; a lid having a closure mechanism that may secure the one or more spa or salon tools within the body portion; an ultraviolet (UV) light mechanism within the body portion that may sanitize the one or more spa or salon tools to kill bacteria, germs, viruses, and/or pathogens that may be present on the one or more spa or salon tools before they are used in a spa or salon; a button on an exterior wall of the sanitizing station, wherein the button may be pressed to activate the UV light mechanism; and a handle and at least one strap to transport the sanitizing station. The sanitizing station may be in a bag form. The closure mechanism may be a zipper. A power supply may provide power to the UV light mechanism. The UV light mechanism may be rechargeable. The UV light mechanism may be recharged through a standard electrical outlet connection or a battery.

Other technical features may be readily apparent to one skilled in the art from the following figures, descriptions and claims.

### BRIEF DESCRIPTION OF THE DRAWINGS

For a more complete understanding of this disclosure, reference is now made to the following description, taken in conjunction with the accompanying drawings, in which:

FIG. 1 depicts an open view of a sanitizing station according to an embodiment of the present disclosure;

FIG. 2 depicts a front closed view of a sanitizing station according to an embodiment of the present disclosure; and

FIG. 3 depicts a rear closed view of a sanitizing station according to an embodiment of the present disclosure.

### DETAILED DESCRIPTION

Embodiments of the present disclosure may provide a portable sanitizing station that may be provided within a salon or spa for cleaning spa and salon tools prior to use on a client/patron. In some embodiments of the present disclosure, a client/patron can bring the sanitizing station to a salon or spa for cleaning their personal spa and salon tools prior to application. Clients can bring the sanitizing station with them when making an appointment at a nail salon or spa. The sanitizing station may ensure that the client's personal spa or salon tools are safe and sanitary to prevent any accumulation of germs, bacteria, and other pathogens from causing illness or infection.

The portable sanitizing station may utilize an ultraviolet (UV) light mechanism provided internally within the body of the sanitizing station to kill any bacteria, germs, viruses, and/or pathogens that may be present on the tools. This may prevent cross-contamination and infection due to unwashed and/or unclean spa and salon tools. It should be appreciated that the UV light mechanism may be rechargeable in some embodiments of the present disclosure. The UV light mechanism may be recharged via a standard electrical outlet connection or a battery, such as a rechargeable universal serial bus (USB) battery. The sanitizing station may include a button or other activation mechanism on the exterior of the sanitizing station that may be pressed or otherwise activated to begin use of the UV light mechanism within the sanitizing station. However, it should be appreciated that the UV light mechanism may be activated through connecting power to the sanitizing station in some embodiments of the present disclosure.

The sanitizing station may be provided as a bag in embodiments of the present disclosure; however, it should be appreciated that the sanitizing station may be presented in other forms without departing from the present disclosure. As discussed in more detail herein, the sanitizing station may include a handle and/or strap(s) that may allow a client/patron to carry the sanitizing station as one might carry a suitcase or a handbag. It should be appreciated that the sanitizing station may be provided in various sizes, styles, and/or designs according to embodiments of the present disclosure.

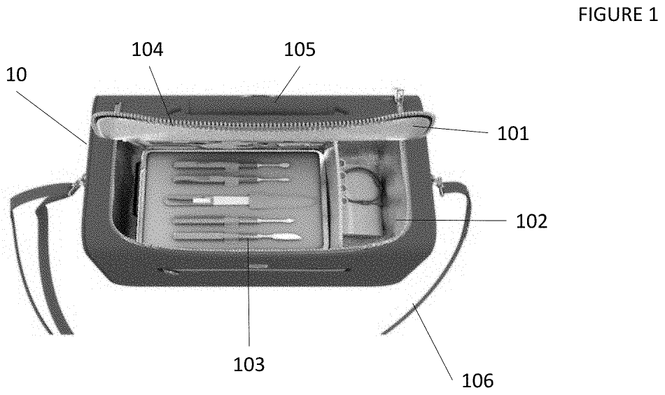

FIG. 1 depicts an open view of sanitizing station 10 according to an embodiment of the present disclosure. As depicted herein, sanitizing station 10 may be provided in a bag form having opening 101 where one or more spa or salon tools 103 may be inserted into body 102. Opening 101 may include closure mechanism 104, such as a zipper. Sanitizing station 10 also may include handle 105 and/or strap 106 so that sanitizing station 10 may be portable for use in more than one location.

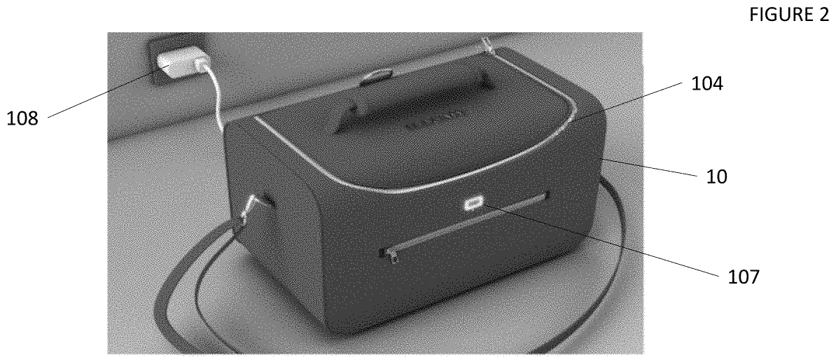

FIG. 2 depicts a front closed view of sanitizing station 10 according to an embodiment of the present disclosure. Closure 104 of sanitizing station 10 is depicted in a closed position. Sanitizing station 10 may include button 107 that may be pressed to activate an ultraviolet (UV) light mechanism inside sanitizing station 10 to sanitize one or more spa or salon tools that have been inserted within sanitizing station 10. It should be appreciated that button 107 may illuminate when the UV light mechanism is in use. Power 108 may be supplied to sanitizing station 10 to illuminate the UV light mechanism when button 107 is pressed. While a button is depicted in FIG. 2, it should be appreciated that other means of UV light activation may be utilized without departing from the present disclosure. For example, the UV light mechanism may be activated through plugging in power 108. In other embodiments of the present disclosure, sanitizing station 10 may be activated through use of battery power.

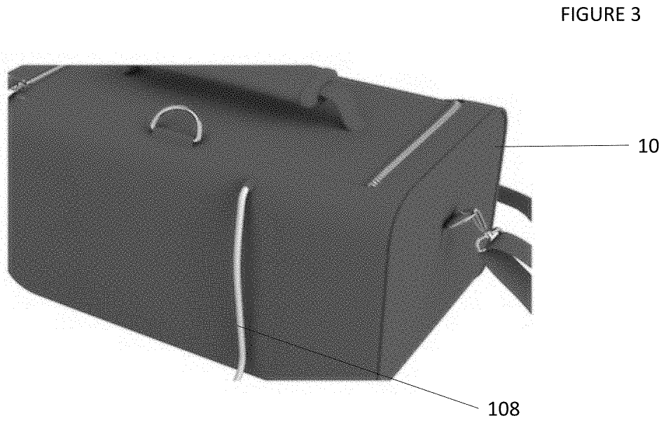

FIG. 3 depicts a rear closed view of sanitizing station 10 according to an embodiment of the present disclosure. As depicted herein, power 108 may include a cord or cable that may be permanently affixed within sanitizing station 10. However, as previously discussed, it should be appreciated that battery power may be employed to utilize sanitizing station 10 without need for power 108 in some embodiments of the present disclosure. It also may be appreciated that power 108 may be removably detachable from sanitizing station 10 in other embodiments of the present disclosure.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A portable sanitizing station for receiving one or more spa or salon tools, the sanitizing station comprising:
a body portion having an opening wherein the one or more spa or salon tools are inserted into the body portion;
a lid having a closure mechanism that secures the one or more spa or salon tools within the body portion; and
an ultraviolet (UV) light mechanism within the body portion that sanitizes the one or more spa or salon tools to kill bacteria, germs, viruses, and/or pathogens that may be present on the one or more spa or salon tools before they are used in a spa or salon.

2. The portable sanitizing station of claim 1, wherein the sanitizing station is in a bag form.

3. The portable sanitizing station of claim 1, wherein the closure mechanism is a zipper.

4. The portable sanitizing station of claim 1 further comprising:
a handle secured to the lid to transport the sanitizing station.

5. The portable sanitizing station of claim 1 further comprising:
one or more straps secured to the sanitizing station to transport the sanitizing station.

6. The portable sanitizing station of claim 1 further comprising:
a button on an exterior wall of the sanitizing station, wherein the button is pressed to activate the UV light mechanism.

7. The portable sanitizing station of claim 1 further comprising:
a power supply that provides power to the UV light mechanism.

8. The portable sanitizing station of claim 1, wherein the UV light mechanism is rechargeable.

9. The portable sanitizing station of claim 8, wherein the UV light mechanism is recharged through a standard electrical outlet connection or a battery.

10. A portable sanitizing station for receiving one or more spa or salon tools, the sanitizing station comprising:
a body portion having an opening wherein the one or more spa or salon tools are inserted into the body portion;
a lid having a closure mechanism that secures the one or more spa or salon tools within the body portion;
an ultraviolet (UV) light mechanism within the body portion that sanitizes the one or more spa or salon tools to kill bacteria, germs, viruses, and/or pathogens that may be present on the one or more spa or salon tools before they are used in a spa or salon; and
a button on an exterior wall of the sanitizing station, wherein the button is pressed to activate the UV light mechanism.

11. The portable sanitizing station of claim 10 further comprising:
a power supply that provides power to the UV light mechanism.

12. The portable sanitizing station of claim 10, wherein the UV light mechanism is rechargeable.

13. The portable sanitizing station of claim 12, wherein the UV light mechanism is recharged through a standard electrical outlet connection or a battery.

14. A portable sanitizing station for receiving one or more spa or salon tools, the sanitizing station comprising:
a body portion having an opening wherein the one or more spa or salon tools are inserted into the body portion;
a lid having a closure mechanism that secures the one or more spa or salon tools within the body portion;
an ultraviolet (UV) light mechanism within the body portion that sanitizes the one or more spa or salon tools to kill bacteria, germs, viruses, and/or pathogens that may be present on the one or more spa or salon tools before they are used in a spa or salon;
a button on an exterior wall of the sanitizing station, wherein the button is pressed to activate the UV light mechanism; and
a handle and at least one strap to transport the sanitizing station.

15. The portable sanitizing station of claim 14, wherein the sanitizing station is in a bag form.

16. The portable sanitizing station of claim 14, wherein the closure mechanism is a zipper.

17. The portable sanitizing station of claim 14 further comprising:
a power supply that provides power to the UV light mechanism.

18. The portable sanitizing station of claim 14, wherein the UV light mechanism is rechargeable.

19. The portable sanitizing station of claim 18, wherein the UV light mechanism is recharged through a standard electrical outlet connection or a battery.
